(19) United States
(12) Patent Application Publication
(10) Pub. No.: US 2010/0229803 A1
(43) Pub. Date: Sep. 16, 2010

(54) ANIMAL DIAPER WITH REDUCED SHIFTING

(76) Inventors: Mark Edward Meissner, San Luis Obispo, CA (US); Kristine Quince Walters, San Luis Obispo, CA (US)

Correspondence Address: Zilka-Kotab, PC, P.O. BOX 721120, SAN JOSE, CA, 95172-1120, US

(21) Appl. No.: 12/404,135

(22) Filed: Mar. 13, 2009

(51) Int. Cl. A01K 23/00; A61F 13/15

(52) U.S. Cl. 119/868; 604/385.09; 604/385.15

(57) ABSTRACT

According to one embodiment, an animal diaper comprises a main body having an upper end, a lower end, an upper portion positioned towards the upper end, a lower portion positioned towards the lower end, an absorbent portion, a tail opening, and an axis extending between the upper and lower ends. The animal diaper also includes first members extending from the lower portion for coupling to the upper portion and second members extending from the lower portion between the first members and the lower end, distal ends of the second members having cooperating fasteners for coupling together. In addition, a spacing between the first and the second members along a wearing animal's back is less than a width of one of the second members measured in a direction parallel to the axis when the wearing animal is standing, and the spacing increases as the wearing animal moves towards a sitting position.

### FIELD OF THE INVENTION

The present invention relates to animal diapers, and more specifically this invention relates to an animal diaper which resists shifting while being worn for prolonged periods of time.

### BACKGROUND

Several attempts have been made at creating an animal diaper that can be worn by various animals, including dogs, cats, weasels, ferrets, rabbits, etc. One problem that persists in prior art animal diapers is that the diaper tends to shift and change positions on the animal while being worn, particularly in response to the animal sitting, jumping, running, and generally moving around as animals tend to do. This shifting and changing of position of the animal diaper can sometimes lead to leaks, spills, and other unwanted side effects of the diaper being out of place. Sometimes, the diaper may fall off completely due to the shifting and loosening of the diaper over time, as well as due to the weight of the diaper after the animal has urinated.

Therefore, it would be beneficial to have an animal diaper that could resist shifting and moving on the animal while being worn.

### SUMMARY OF THE INVENTION

According to one embodiment, an animal diaper comprises a main body having an upper end, a lower end, an upper portion positioned towards the upper end, a lower portion positioned towards the lower end, an absorbent portion, a tail opening, and an axis extending between the upper and lower ends. The animal diaper also includes first members extending from the lower portion for coupling to the upper portion and second members extending from the lower portion between the first members and the lower end, distal ends of the second members having cooperating fasteners for coupling together. In addition, a spacing between the first and the second members along a wearing animal's back is less than a width of one of the second members measured in a direction parallel to the axis when the wearing animal is standing, and the spacing increases as the wearing animal moves towards a sitting position.

In another embodiment, an animal diaper includes a main body having an upper end, a lower end, an upper portion positioned towards the upper end, a lower portion positioned towards the lower end, an absorbent portion, a tail opening, and an axis extending between the upper and lower ends. Also, the animal diaper includes first members extending from the lower portion for coupling to the upper portion and second members extending from the lower portion between the first members and the lower end, distal ends of the second members having cooperating fasteners for coupling together. In addition, widths of the second members measured in a direction parallel to the axis is greater than ¼ of a minimum width of the main body measured in a direction perpendicular to the axis.

An animal diaper according to another embodiment includes a main body having an tipper end, a lower end, an tipper portion positioned towards the upper end, a lower portion positioned towards the lower end, an absorbent portion, a tail opening, and an axis extending between the upper and lower ends. Also, the animal diaper includes first members extending from the lower portion for coupling to the upper portion and second members extending from the lower portion between the first members and the lower end, distal ends of the second members having cooperating fasteners for coupling together. Also, a spacing between the first and the second members along a wearing animal's back is less than a width of one of the second members measured in a direction parallel to the axis when the wearing animal is standing, and the spacing increases as the wearing animal moves towards a sitting position. In addition, the lower portion includes an elastic portion positioned towards the lower end, and the first and second members remain at about the same distance from a center of pivot of a lower half of the backbone of the wearing animal when the wearing animal is standing and when the wearing animal is sitting. Furthermore, the second members are positioned at least 60% of a distance between the wearing animal's shoulders and hips from the wearing animal's shoulders, and widths of the second members measured in a direction parallel to the axis is greater than ¼ of a minimum width of the main body measured in a direction perpendicular to the axis.

Other aspects, advantages and embodiments of the present invention will become apparent from the following detailed description, which, when taken in conjunction with the drawings, illustrate by way of example the principles of the invention.

### BRIEF DESCRIPTION OF THE DRAWINGS

For a fuller understanding of the nature and advantages of the present invention, as well as the preferred mode of use, reference should be made to the following detailed description read in conjunction with the accompanying drawings.

FIG. 1 is a schematic diagram of an animal diaper according to one embodiment.

FIG. 2 is a schematic diagram of an animal diaper according to one embodiment.

FIG. 3 is a schematic diagram of an animal diaper shown on a dog according to one embodiment.

FIG. 4 is a schematic diagram of an animal diaper shown on a dog according to one embodiment.

### DETAILED DESCRIPTION

The following paragraphs describe certain features and combinations of features that can be used in connection with the invention and each embodiment, as generally described below. Also, particular features described hereinafter can be used in combination with other described features in each of the various possible combinations and permutations. As such, the invention is not limited to the specifically described embodiments.

Unless otherwise specifically defined herein, all terms are to be given their broadest possible interpretation and scope including one or more meanings implied from the specification as well as one or more meanings understood by those skilled in the art and/or as defined in dictionaries, treatises, etc.

The following description is made for the purpose of illustrating the general principles of the present invention and is not meant to limit the inventive concepts claimed herein. Further, particular features described herein can be used in combination with other described features in each of the various possible combinations and permutations.

It must also be noted that, as used in the specification and the appended claims, the singular forms “a,” “an” and “the” include plural referents unless otherwise specified.

According to one general embodiment, an animal diaper includes a main body having an upper end, a lower end, an upper portion positioned towards the upper end, a lower portion positioned towards the lower end, an absorbent portion, a tail opening, and an axis extending between the upper and lower ends; first members extending from the lower portion for coupling to the upper portion; and second members extending from the lower portion between the first members and the lower end, distal ends of the second members having cooperating fasteners for coupling together; wherein a spacing between the first and the second members along a wearing animal's back is less than a width of one of the second members measured in a direction parallel to the axis when the wearing animal is standing, wherein the spacing increases as the wearing animal moves towards a sitting position.

In another general embodiment, an animal diaper includes a main body having an upper end, a lower end, an upper portion positioned towards the upper end, a lower portion positioned towards the lower end, an absorbent portion, a tail opening, and an axis extending between the upper and lower ends; first members extending from the lower portion for coupling to the upper portion; and second members extending from the lower portion between the first members and the lower end, distal ends of the second members having cooperating fasteners for coupling together; wherein widths of the second members measured in a direction parallel to the axis is greater than ¼ of a minimum width of the main body measured in a direction perpendicular to the axis.

In another general embodiment, an animal diaper includes a main body having an upper end, a lower end, an upper portion positioned towards the upper end, a lower portion positioned towards the lower end, an absorbent portion, a tail opening, and an axis extending between the upper and lower ends; first members extending from the lower portion for coupling to the upper portion; and second members extending from the lower portion between the first members and the lower end, distal ends of the second members having cooperating fasteners for coupling together; wherein a spacing between the first and the second members along a wearing animal's back is less than a width of one of the second members measured in a direction parallel to the axis when the wearing animal is standing, wherein the spacing increases as the wearing animal moves towards a sitting position, wherein the lower portion includes an elastic portion positioned towards the lower end, wherein the first and second members remain at about the same distance from a center of pivot of a lower half of the backbone of the wearing animal when the wearing animal is standing and when the wearing animal is sitting, wherein the second members are positioned at least 60% of a distance between the wearing animal's shoulders and hips from the wearing animal's shoulders, wherein widths of the second members measured in a direction parallel to the axis is greater than ¼ of a minimum width of the main body measured in a direction perpendicular to the axis.

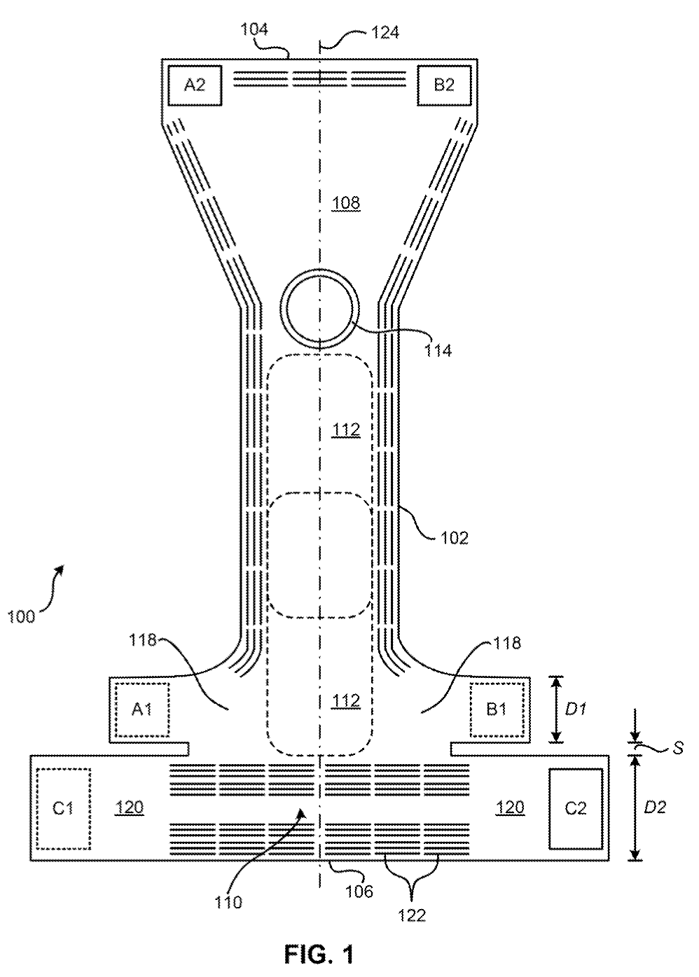

Now referring to FIG. 1, an animal diaper 100 is shown according to one embodiment. The animal diaper 100 includes a main body 102 having an upper end 104 that generally extends over an animal's tailbone, a lower end 106 that generally extends towards an animal's underside, an upper portion 108 positioned towards the upper end 104, a lower portion 110 positioned towards the lower end 106, an absorbent portion 112, a tail opening 114 (which may be a hole, flap, split, etc.) and an axis 124 extending between the upper end 104 and lower end 106. Also, the animal diaper 100 includes first members 118 extending from the lower portion 110 for coupling to the upper portion 108, and second members 120 extending from the lower portion 110 between the first members 118 and the lower end 106, distal ends of the second members 120 having cooperating fasteners for coupling together.

In preferred embodiments, the animal diaper 100 may be designed so that it resists shifting during wearing by an animal. In addition, the animal diaper 100 is intended to be comfortable to wear for the animal, such that the animal is not apt to remove the diaper 100 through scratching, rubbing, etc. To achieve these advantages, in one approach, a spacing S between the first member 118 and the second member 120 along a wearing animal's back is less than a width of one of the second members 120 measured in a direction parallel to the axis when the wearing animal is standing. To describe this distance S in another way, S is less than D1 and/or D2. Also, the spacing S increases as the wearing animal moves towards a sitting position.

This close spacing along with the wide second members 120 is critical in some embodiments to resist shifting of the animal diaper 100 during wearing by an animal, as the width tends to keep the second member above the fur of the animal, which tends to point backwards and which would otherwise create a ratchet effect urging the diaper towards the back of the animal. Moreover, the width of the second members 120 relative to the distance S is also critical for the comfort of the animal, as a thinner strap would tend to dig into the animal's fur, causing discomfort and possibly causing the animal attempt to alleviate the discomfort by scratching and/or chewing, which in turn would tend to cause the diaper to become removed.

In additional preferred embodiments, the dimensions of the animal diaper 100 may be increased and/or decreased in overall size to accommodate different sized animals. For example, a diaper intended for a Labrador Retriever may be larger in overall size than a diaper intended for a Shih Tzu. In another example, a diaper intended to be worn by a rabbit may be smaller than a diaper intended to be worn by a Great Dane.

Also, in some embodiments, the dimensions of some of the portions of the animal diaper 100 may be longer and/or shorter depending on the type and/or size of animal that is intended to wear the diaper. For example, if the diaper is intended to be worn by a ferret, the distance between C1 and C2 may be relatively small compared to the distance between C1 and C2 for a diaper intended to be worn by a Malamute. However, the distance between A1 and A2 (and also B1 and B2, which generally will be the same dimension) will be relatively long for the diaper intended to be worn by a ferret when compared to a diaper intended to be worn by a malamute. This is due to the ferret having a long, slender body, and a malamute having a huskier frame.

In some embodiments, the main body 102 may be disposable. This may allow a new disposable diaper to be used each time an animal diaper is to be used. Such a diaper may include components and/or layers that are similar to disposable diapers for human infants or adult diapers for human adults. For example, the absorbent portion 112 of the diaper may be formed from wood pulp and/or other absorbent materials. In one particularly preferred embodiment, the absorbent portion 112 includes superabsorbent polymers. The outer layer of the diaper may provide a liquid-proof barrier to keep moisture contained.

In other embodiments, the main body 102 may be washable and/or cleanable. For example, the main body 102 may be able to be put through a washing machine for clothes, may be hand washable, may be dry-cleanable, etc.

In some approaches, the absorbent portion 112 may be removable and/or replaceable, e.g., the absorbent portion, since it normally will receive and contain the fecal matter and/or urine of the wearing animal, may be removed and replaced with a new absorbent portion or with a cleaned absorbent portion. Any known coupling mechanism may be used to couple a removable and/or replaceable absorbent portion 112, including hooks and loops fasteners, snaps, adhesive, a pouch into which the absorbent portion 112 is inserted, etc.

In some further approaches, the absorbent portion 112 may be washable and/or cleanable. In this fashion, when an absorbent portion becomes soiled, it can be removed and cleaned for later use. Moreover, where the absorbent portion 112 is integrally formed with the diaper, i.e., is nonremovable, the entire diaper may be washable and/or cleanable.

In other embodiments, the absorbent portion 112 may be integral to the main body 102, e.g., when the absorbent portion 112 becomes soiled, the absorbent portion 112 and the main body 102 may be disposed of together and replaced by a new animal diaper 100.

In more embodiments, the absorbent portion 112 may be in a position closer to the tail opening 114 when the diaper 100 is to be placed on a female animal. In other embodiments, the absorbent portion 112 may be in a position farther from the tail opening 114 when the diaper 100 is to be placed on a male animal. In even more embodiments, a larger absorbent portion 112 may be used, which may be positioned to cover both areas, thereby not requiring moving the absorbent portion or replacing the absorbent portion depending on the sex of the animal.

Note that the shape of the absorbent portion 112 is provided by way of example only, and the absorbent portion 1112 in a given embodiment may have a shape similar to that shown, or any other shape. Moreover, the absorbent portion may extend across a larger or smaller area than that shown, and in some approaches may extend across a majority of the area of the diaper.

The first members 118 and second members 120 may include any type of fasteners, such as hooks and loops fasteners, snaps, buttons, D-rings, etc. Of course, if the fastener type included has different portions, then the proper portions may be provided on each corresponding fastener, e.g., A1 has a corresponding fastener to A2, B1 has a corresponding fastener to B2, etc. In FIG. 1, A1, B1, and C1 are shown on the underside of the diaper 100, e.g., the opposite side of the diaper from A2, B2, and C2. Of course, the fasteners may be in any location, as long as they still allow the diaper 100 to be placed on an animal, and can hold the diaper in place over a period of time.

According to some preferred embodiments, the lower portion 110 may include an elastic portion 122 (or some other stretchable material, which could give elasticity to the members of the diaper) positioned towards the lower end 106. In more embodiments, elastic portions may be located around the edges of all portions of the animal diaper 100, such as the upper portion 108, upper end 104, lower portion 110, etc.

In various embodiments, the animal diaper 100 may be comprised of one or more layers of man-made and/or natural materials. For example, one or more layers may include a material which is resiliently deformable, such as Lycra, Spandex, etc. In these embodiments, the animal diaper 100 may or may not include elastic and/or stretchable portions. In other embodiments, the animal diaper 100 may be comprised of a scent resistant material; a washable material such as cotton, polyester, etc.; a long lasting material such as a polymer-containing fabric; a material which tends to grip animal fur such as a rubber, adhesive, etc.; a fluid barrier material, e.g., a plastic forming an outer layer of the animal diaper 100; etc. In various embodiments, the absorbent portion 112 of the animal diaper 100 may be formed from wood pulp, cotton, and/or other absorbent materials. In one particularly preferred embodiment, the absorbent portion 112 includes superabsorbent polymers.

According to some embodiments, the second members 120 may be positioned between a wearing animal's shoulders and hips. Furthermore, the second members 120 may be positioned at least 60% of the way from a distance between the wearing animal's shoulders and hips, closer to the wearing animal's hips. In other embodiments, the second members 120 may be positioned at least 70% of the way or more toward the wearing animal's hips. Of course, the second members 120 may also be located closer to the wearing animal's shoulders, according to some other embodiments.

The unique placement of the second members 120 may allow the first members 118 and the second members 120 to remain at about the same distance from a center of pivot of a lower half of the backbone of the wearing animal when the wearing animal is standing and when the wearing animal is sitting. This in turn may prevent parts of the diaper 100 from shifting when the animal transitions between standing and sitting, thereby keeping the diaper on the animal and positioned properly to absorb discharges from the wearing animal.

In some approaches, the first members 118 and second members 120 may be designed to contact each other along the wearing animal's back when the wearing animal is standing. In some other embodiments, the first members 118 and second members 120 may be designed to not contact each other along the wearing animal's back when the wearing animal is standing, possibly enhancing the diaper's 100 resistance to shifting while being worn by an animal.

According to some preferred approaches, widths of the second members 120 measured in a direction parallel to the axis 124 may be greater than ¼ of a minimum width of the main body 102 measured in a direction perpendicular to the axis 124. Of course, the second members 120 may be wider or narrower than ¼ of a minimum width of the main body 102 measured in a direction perpendicular to the axis 124.

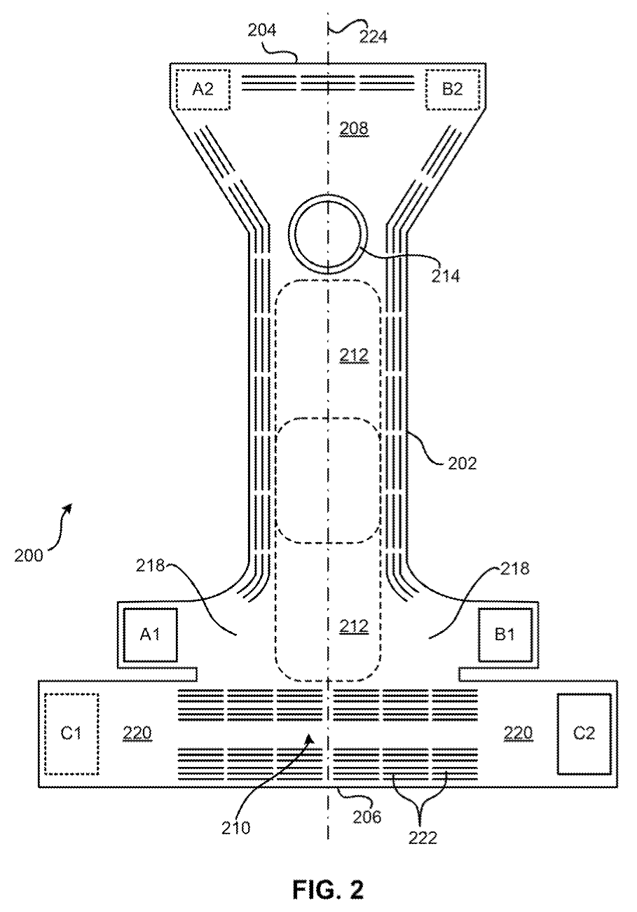

Now referring to FIG. 2, according to one embodiment, an animal diaper 200 includes a main body 202 having an upper end 204, a lower end 206, an upper portion 208 positioned towards the upper end 204, a lower portion 210 positioned towards the lower end 206, an absorbent portion 212, a tail opening 214 which can be a hole, flap, etc., and an axis 224 extending between the upper end 204 and lower end 206. The animal diaper 200 also includes first members 218 extending from the lower portion 210 for coupling to the upper portion 208 and second members 220 extending from the lower portion 210 between the first members 218 and the lower end 210, distal ends of the second members 220 having cooperating fasteners for coupling together. Also, widths of the second members 220 measured in a direction parallel to the axis 224 is greater than ¼ of a minimum width of the main body 202 measured in a direction perpendicular to the axis 224. Of course, any of the previous descriptions may apply to this embodiment as well.

In FIG. 2, the diaper 200 is shown with a shorter upper portion 208 than the diaper shown in FIG. 1. This may enable the diaper 200 to resist shifting when being worn by an animal, especially if the animal is smaller in stature.

In some embodiments, the main body 202 may be disposable. This may allow a new disposable diaper to be used each time an animal diaper is to be used, similar to disposable diapers for human infants or adult diapers for human adults.

In other embodiments, the main body 202 may be washable and/or cleanable. For example, the main body 202 may be able to be put through a washing machine for clothes, may be hand washable, may be dry-cleanable, etc.

In some approaches, the absorbent portion 212 may be removable and/or replaceable, e.g., the absorbent portion, since it normally will receive and contain the fecal matter and/or urine of the wearing animal, may be removed and replaced with a new absorbent portion or with a cleaned absorbent portion. In some further approaches, the absorbent portion 212 may be washable and/or cleanable. In this fashion, when an absorbent portion becomes soiled, it can be removed and cleaned for use later.

In other embodiments, the absorbent portion 212 may be integral to the main body 202, e.g., when the absorbent portion 212 becomes soiled, the absorbent portion 212 and the main body 202 may be disposed of together, and replaced by a new animal diaper 200.

In more embodiments, the absorbent portion 212 may be in a position closer to the tail opening 214 when the diaper 200 is to be placed on a female animal. In other embodiments, the absorbent portion 212 may be in a position farther from the tail opening 214 when the diaper 200 is to be placed on a male animal. In even more embodiments, a larger absorbent portion 212 may be used, which may be positioned to cover both areas, thereby not requiring moving the absorbent portion or replacing the absorbent portion depending on the sex of the animal.

The first members 218 and second members 220 may include any type of fasteners, such as hooks and loops fasteners, snaps, buttons, D-rings, etc. Of course, if the fastener type included has different portions, then the proper portions may be provided on each corresponding fastener, e.g., A1 has a corresponding fastener to A2, B1 has a corresponding fastener to B2, etc. Also, as shown in FIG. 2, A2, B2, and C1 are shown on the underside of the diaper 200, e.g., the opposite side of the diaper from A1, B1, and C2. Of course, the fasteners may be in any location, as long as they still allow the diaper 200 to be placed on an animal, and can hold the diaper in place over a period of time.

According to some preferred embodiments, the lower portion 210 may include an elastic portion 222 (or some other stretchable material, which could give elasticity to the members of the diaper) positioned towards the lower end 206. In more embodiments, elastic portions may be located around the edges of all portions of the animal diaper 200, such as the upper portion 208, upper end 204, lower portion 210, etc.

In various embodiments, the animal diaper 200 may be comprised of one or more layers of man-made and/or natural materials. For example, one or more layers may include a material which is resiliently deformable, such as Lycra, Spandex, etc. In these embodiments, the animal diaper 200 may or may not include elastic and/or stretchable portions. In other embodiments, the animal diaper 200 may be comprised of a scent resistant material; a washable material such as cotton, polyester, etc.; a long lasting material such as a polymer-containing fabric; a material which tends to grip animal fur such as a rubber, adhesive, etc.; a fluid barrier material, e.g., a plastic forming an outer layer of the animal diaper 200; etc. In various embodiments, the absorbent portion 212 of the animal diaper 200 may be formed from wood pulp, cotton, and/or other absorbent materials. In one particularly preferred embodiment, the absorbent portion 212 includes superabsorbent polymers.

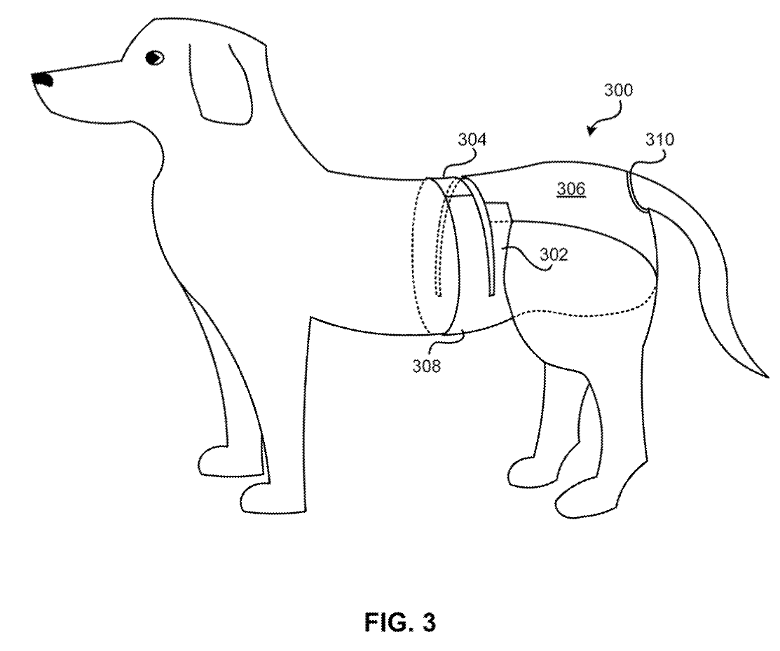

Now referring to FIG. 3, an animal diaper 300 is shown on an animal. In FIG. 3, the animal diaper 300 is shown on a dog, but it can be used on any animal with substantially the same orientation of tail/legs/urination, such as cats, rabbits, weasels, ferrets, pigs, etc. The animal diaper 300 may have any desired configuration, including those described above.

The diaper 300 is shown in place on an animal, and includes first members 302 attached to the upper portion 306. Also, second members 304 are attached to one another, while a tail protrudes through a tail opening 310. The lower portion 308 is in place under the animal, where an absorbent portion (not shown) can receive any fecal matter and/or urine from the animal.

According to some embodiments, the animal diaper 300 may be placed on the animal such that the first members 302 may be at least partially held in place by the proximity to the animal's hind legs and/or hips. For example, the first members 302 may partially contact the hind legs to keep them in a forward position, and to reduce slack in the animal diaper 300 near the animal's tail.

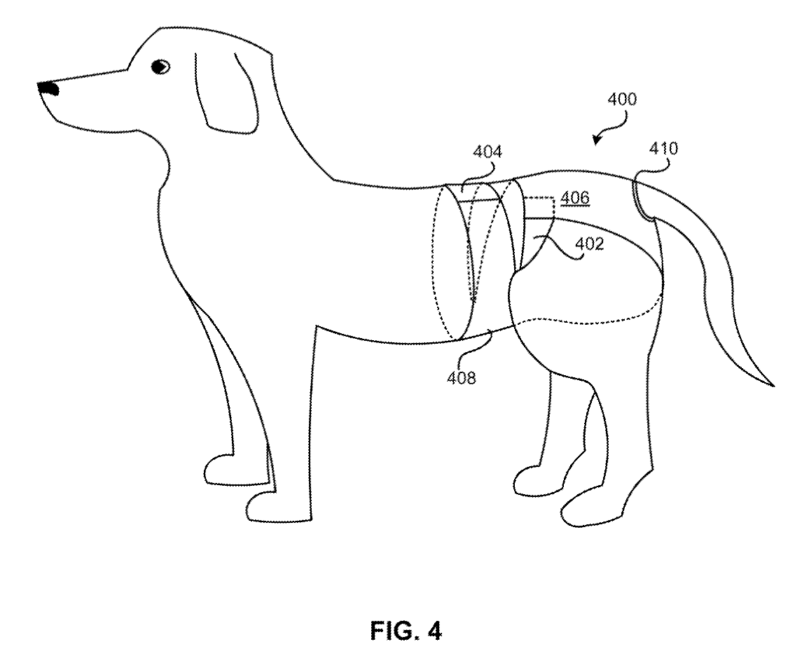

In reference to FIG. 4, an animal diaper 400 is shown according to one embodiment. The diaper 400 may include first members 402 and second members 404, where the spacing between where the first members 402 attach to the upper portion 406 may be greater than the spacing shown in FIG. 3. The animal diaper 400 may have any desired configuration, including those described above.

Also, the length of the upper portion 406 may be shorter relative to the tail opening 410 than that shown in FIG. 3. In the embodiment of FIG. 4, the shorter length of the upper portion 406 is critical for causing the first members 402 to hug closely to the hips and thighs of the animal, such that the animal's legs urge the diaper 400 forward, thereby preventing the formation of a gap between the diaper and the hind end of the animal. Prevention of such a gap is critical to keeping fecal matter and/or urine contained in the animal diaper 400.

According to some embodiments, the animal diaper 400 may be placed on the animal such that the first members 402 may be at least partially held in place by the proximity to the animal's hind legs and/or hips. For example, the first members 402 may partially contact the hind legs to keep them in a forward position, and to reduce slack in the animal diaper 400 near the animal's tail. Also, the shorter length of the upper portion 406 as compared to the animal diaper 300 shown in FIG. 3 may enhance the diaper's ability to stay in place while the animal moves, sits, runs, etc.

While various embodiments have been described above, it should be understood that they have been presented by way of example only, and not limitation. Thus, the breadth and scope of an embodiment should not be limited by any of the above-described exemplary embodiments, but should be defined only in accordance with the following claims and their equivalents.

What is claimed is:

1. An animal diaper, comprising:
a main body having an upper end, a lower end, an upper portion positioned towards the upper end, a lower portion positioned towards the lower end, an absorbent portion, a tail opening, and an axis extending between the upper and lower ends;
first members extending from the lower portion for coupling to the upper portion; and
second members extending from the lower portion between the first members and the lower end, distal ends of the second members having cooperating fasteners for coupling together;
wherein a spacing between the first and the second members along a wearing animal's back is less than a width of one of the second members measured in a direction parallel to the axis when the wearing animal is standing,
wherein the spacing increases as the wearing animal moves towards a sitting position.

2. The animal diaper of claim 1, wherein the main body is disposable.

3. The animal diaper of claim 1, wherein the main body is washable.

4. The animal diaper of claim 1, wherein the absorbent portion is removable.

5. The animal diaper of claim 4, wherein the absorbent portion is washable.

6. The animal diaper of claim 1, wherein the lower portion includes an elastic portion positioned towards the lower end.

7. The animal diaper of claim 1, wherein the second members are positioned at least 60% of a distance between the wearing animal's shoulders and hips from the wearing animal's shoulders.

8. The animal diaper of claim 1, wherein the first and second members remain at about the same distance from a center of pivot of a lower half of the backbone of the wearing animal when the wearing animal is standing and when the wearing animal is sitting.

9. The animal diaper of claim 1, wherein the first and second members are designed to contact each other along the wearing animal's back when the wearing animal is standing.

10. The animal diaper of claim 1, wherein widths of the second members measured in a direction parallel to the axis is greater than ¼ of a minimum width of the main body measured in a direction perpendicular to the axis.

11. An animal diaper, comprising:
a main body having an upper end, a lower end, an upper portion positioned towards the upper end, a lower portion positioned towards the lower end, an absorbent portion, a tail opening, and an axis extending between the upper and lower ends;
first members extending from the lower portion for coupling to the upper portion; and
second members extending from the lower portion between the first members and the lower end, distal ends of the second members having cooperating fasteners for coupling together;
wherein widths of the second members measured in a direction parallel to the axis is greater than ¼ of a minimum width of the main body measured in a direction perpendicular to the axis.

12. The animal diaper of claim 11, wherein the main body is disposable.

13. The animal diaper of claim 11, wherein the main body is washable.

14. The animal diaper of claim 11, wherein the absorbent portion is removable.

15. The animal diaper of claim 14, wherein the absorbent portion is washable.

16. The animal diaper of claim 11, wherein the lower portion includes an elastic portion positioned towards the lower end.

17. The animal diaper of claim 11, wherein the first and second members remain at about the same distance from a center of pivot of a lower half of the backbone of the wearing animal when the wearing animal is standing and when the wearing animal is sitting.

18. The animal diaper of claim 11, wherein the first and second members are designed to contact each other along the wearing animal's back when the wearing animal is standing.

19. The animal diaper of claim 11, wherein the second members are positioned at least 60% of a distance between the wearing animal's shoulders and hips from the wearing animal's shoulders.

20. An animal diaper, comprising:
a main body having an upper end, a lower end, an upper portion positioned towards the upper end, a lower portion positioned towards the lower end, an absorbent portion, a tail opening, and an axis extending between the upper and lower ends;
first members extending from the lower portion for coupling to the upper portion; and
second members extending from the lower portion between the first members and the lower end, distal ends of the second members having cooperating fasteners for coupling together;
wherein a spacing between the first and the second members along a wearing animal's back is less than a width of one of the second members measured in a direction parallel to the axis when the wearing animal is standing,
wherein the spacing increases as the wearing animal moves towards a sitting position,
wherein the lower portion includes an elastic portion positioned towards the lower end,
wherein the first and second members remain at about the same distance from a center of pivot of a lower half of the backbone of the wearing animal when the wearing animal is standing and when the wearing animal is sitting,
wherein the second members are positioned at least 60% of a distance between the wearing animal's shoulders and hips from the wearing animal's shoulders,
wherein widths of the second members measured in a direction parallel to the axis is greater than ¼ of a minimum width of the main body measured in a direction perpendicular to the axis.

21. The animal diaper of claim 20, wherein the main body is disposable.

22. The animal diaper of claim 20, wherein the main body is washable.

23. The animal diaper of claim 20, wherein the absorbent portion is removable.

24. The animal diaper of claim 23, wherein the absorbent portion is washable.
